(19) United States
(12) Patent Application Publication
(10) Pub. No.: US 2007/0208397 A1
(43) Pub. Date: Sep. 6, 2007

(54) DEVICE AND METHOD OF PHOTOTHERAPY FOR JAUNDICED INFANTS

(76) Inventor: Steven Gardner, Columbia, SC (US)

Correspondence Address: BROOKS KUSHMAN P.C., 1000 TOWN CENTER, TWENTY-SECOND FLOOR, SOUTHFIELD, MI, 48075, US

(21) Appl. No.: 11/744,963

(22) Filed: May 7, 2007

Related U.S. Application Data
(63) Continuation-in-part of application No. PCT/US05/40399, filed on Nov. 9, 2005.
(60) Provisional application No. 60/626,169, filed on Nov. 9, 2004.

(51) Int. Cl. A61N 5/06

(52) U.S. Cl. 607/88

(57) ABSTRACT

A phototherapy device and method for treating neonatal hyperbilirubinemia jaundice) and related conditions. The device comprises a flexible material encasing flexible circuitry that allows the device to flex as an infant is positioned on it, while, also protecting the circuitry. The circuitry comprises a plurality of light emitting diodes (LEDs) mounted within flexible circuitry, means for altering the duty cycle of the LEDs, and wiring to connect the circuitry to a power supply.

### CROSS-REFERENCE TO RELATED APPLICATIONS

This application is a Continuation-in-part of PCT/US2005/040399 filed Nov. 9, 2005, which claims the benefit of U.S. provisional application Ser. No. 60/626,169 filed Nov. 9, 2004.

### BACKGROUND OF THE INVENTION

1. Field of the Invention

This invention relates generally to the treatment of neonatal hyperbilirubinemia jaundice), and more specifically it relates to phototherapy treatment methods and devices.

2. Background Art

Approximately 60% of infants born in the United States each year become clinically jaundiced. Jaundice, or hyperbilirubinemia, results from increased production and transiently impaired elimination of bilirubin. While most affected neonates recover rapidly, some infants show persistently high levels of unconjugated bilirubin. Such high levels can lead to kernicterus, a condition involving deposition of bilirubin in the brain, which leads to deficits in cognition, neuromuscular tone and control, hearing, and even death. The most common therapy for neonatal hyperbilirubinemia is phototherapy. It is estimated that as many as 400,000 neonates in the United States receive phototherapy every year. Phototherapy facilitates the transformation of unconjugated bilirubin to compounds that are more easily excreted.

Phototherapy for treating hyperbilirubinemia is commonly delivered using fluorescent lamps suspended above the neonate. However, fluorescent lamps generate heat (infrared radiation), which prevents their placement close to the infant, thereby decreasing the irradiance. Fluorescent light is of a broad spectral range, and cannot be produced in the narrow wavelength range desired. Conventional phototherapy devices typically illuminate the newborn only from above, and do not therefore make optimal use of the available skin area.

The use of fluorescent lamps for phototherapy leads to adverse side effects in many newborns. Such side effects include increased insensible water loss, hypothermia, loose and frequent bowel movements, tanning, and potential nasal obstruction by the eye pads required for preventing retinal damage. Furthermore, there are concerns that phototherapy using fluorescent lamps has potentially harmful effects on biological rhythms, and may increase the incidence of skin cancer in neonates subject to repeated treatment.

### SUMMARY OF THE INVENTION

The present invention provides a phototherapy device and method for treating neonatal hyperbilirubinemia jaundice) and related conditions, such as Crigler-Najjar Syndrome. The present invention is an improved phototherapy device and method for treating neonatal hyperbilirubinemia. The device comprises a flexible material that encases (potted) flexible circuitry which allows an infant to be held and carried by a caregiver while the infant is undergoing treatment. The circuitry comprises a plurality of light emitting diodes (LEDs) mounted in a pattern, flexible circuitry, means to alter the duty cycle of the LEDs, and wiring to connect the circuitry to a power supply, all of which, in the present invention, are fully potted/encased in the flexible material.

### BRIEF DESCRIPTION OF THE DRAWINGS

FIG. 1 is an exploded view of the larger elements of the device, with an infant in position for use;

FIG. 2 is an exploded view demonstrated the device as used with an infant;

FIG. 3 is a top plan view of the LED light panel;

FIG. 4 is a side view of the LED light panel;

FIG. 5 is a side view of an additional embodiment of the LED light panel; and

FIG. 6 is an alternative embodiment of a LED light panel detailing the potted circuitry.

### DETAILED DESCRIPTION OF THE PREFERRED EMBODIMENT(S)

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for the claims and/or as a representative basis for teaching one skilled in the art to utilize the present invention.

Although the following detailed description contains many specifics for the purposes of illustration, and variations and alterations to the following details are within the scope of the invention. Accordingly, the following embodiments of the invention are set forth without any loss of generality to, and without imposing limitations upon, the claimed invention.

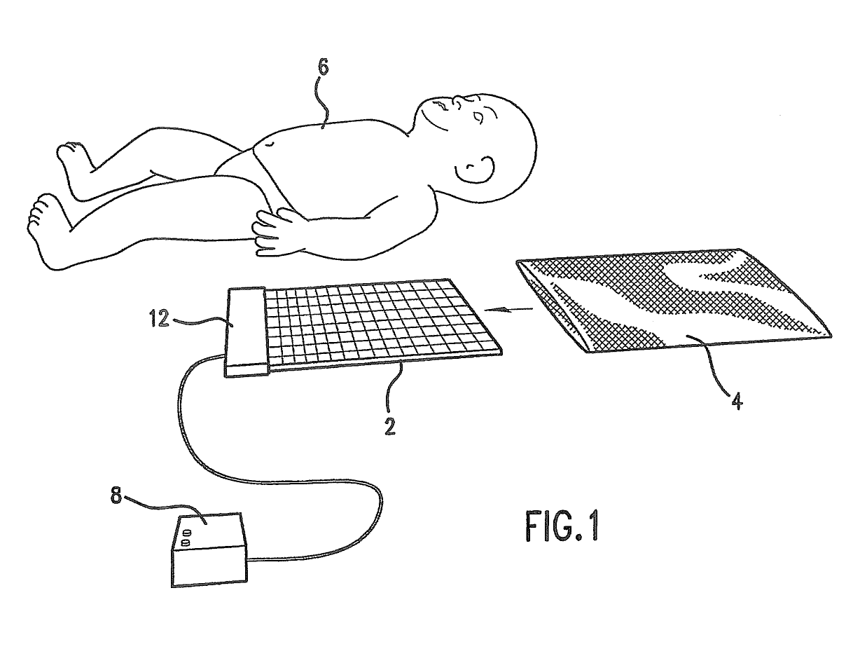

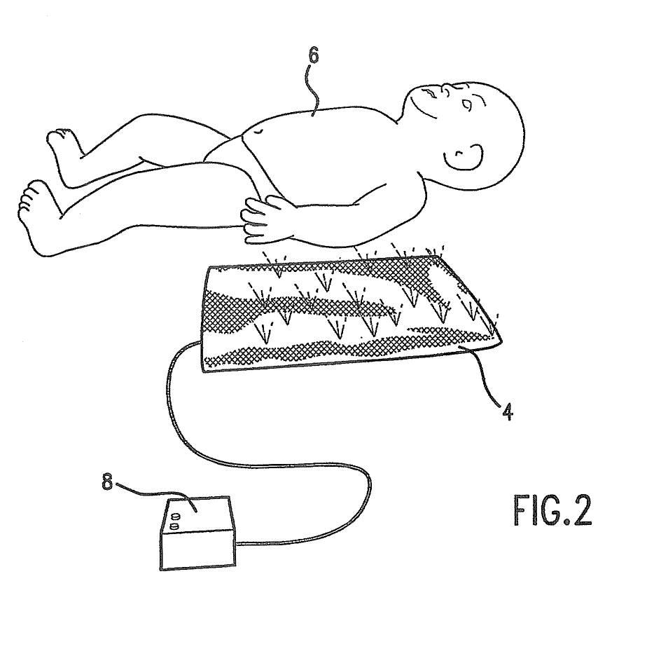

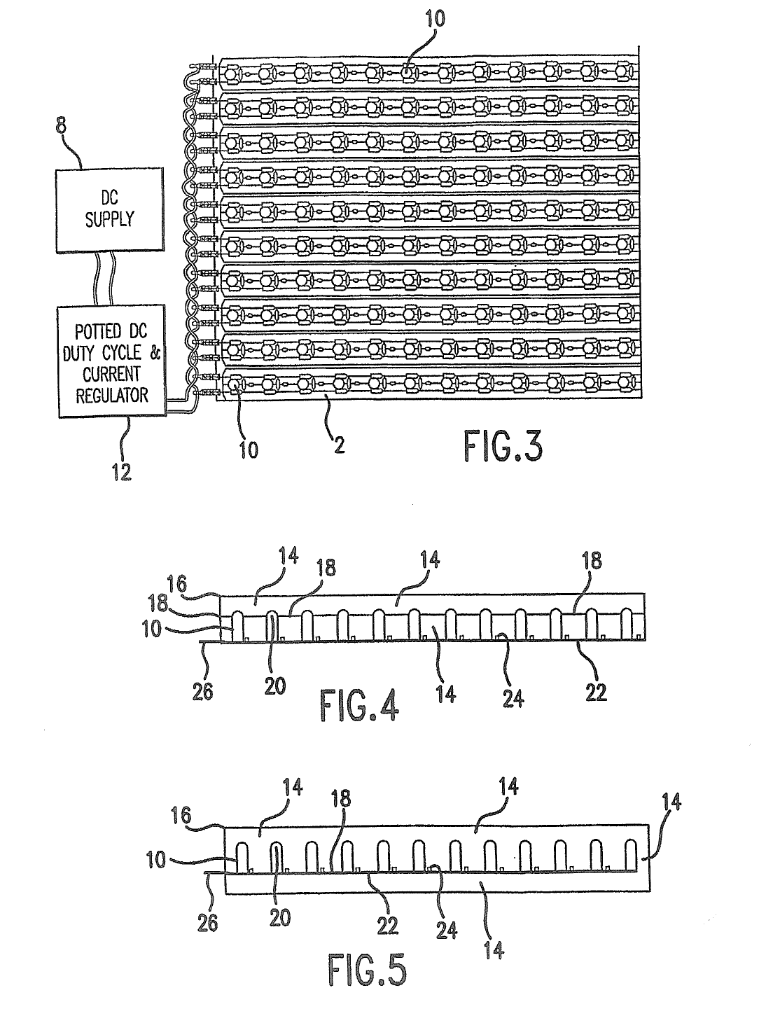

FIG. 1 shows the phototherapy panel 2 with a cover 4. The panel is preferred to be slightly larger than the length of the back of an infant 6. A power supply unit 8 powers the device. As shown in FIG. 2, the sheath allows the light transmitted from the panel to be transmitted through the sheath to the infant's back.

In one embodiment, the phototherapy panel comprises a flexible backing material, a transparent liner, and a flexible circuitry substrate, with light emitting diodes (LEDs) mounted to the flexible circuitry substrate and conductively connected to a power supply. An infant 6 is placed over the panel, with the LEDs emitting light toward the infant's back. The panel is adapted to provide light exposure by means of the LEDs over 100% of the infant's back, or somewhat beyond the infant's back. The phototherapy panel will effectively treat neonatal hyperbilirubinemia, also known as jaundice, via phototherapy.

It is preferred that the portion of the panel 2 in which LEDs are present is wider and longer than the infant's back. However, the panel is preferred to not be substantially longer or wider than is necessary to provide phototherapy to the infant's back. The portion of the panel comprising LED's (the treatment area) is preferred to not be larger than 12 cm by 18 cm. The thickness of the panel is preferred to be 1-2 cm.

The panel as constructed is preferred to be easily portable, that is, the infant may be carried with panel in place against the infant's back. The infant may be comforted and/or transported by a caregiver while the device is in use. Accordingly, the device is constructed so that it is as small as possible, while still providing efficacy. Further, the device as constructed in the preferred embodiment does not need cooling, such as by fans, fins, channels, or any other heat removal method or device, but it is preferred to keep the number of LEDs to a minimum number that is efficacious so that heat is not detrimental to the device or the infant.

In the preferred embodiment, the LEDs are preferred to emit high-intensity blue light suitable for treating neonatal hyperbilirubinemia. In another embodiment, the LEDs can be arranged in groups of different colors so that the color balance can be modified via the control circuitry varying the relative power to the LED groups to facilitate greater efficacy or the treatment of other medical conditions that can benefit from phototherapy. The LEDs are arranged to optimize intensity and coverage. The unit is preferred to have not fewer than one LED per 2.5 square centimeter within region of the matrix where LEDs are present.

A Power supply 8 is provided. The power supply can be an AC power supply and/or a DC power supply for higher degrees of portability. The portable power supply may be one or more batteries that supply direct current to the LEDs, and enable the unit to be highly portable. The portable power supply is low voltage direct current in the preferred embodiment, thereby reducing the risk of harmful electric shock to the patient. The AC power supply will use a standard transformer plug to reduce the power level and match that of the DC power supply and may be combined with a battery charger current and battery pouch pack to render the unit portable. Silicon chips enable the LEDs to have their duty cycle altered from 100% actuation time to as little as 10%, thereby increasing battery life, as well as decreasing the amount of heat generated.

The invention provides doctors and parents with a new device that combines the benefits of a fiber-optic panel and a phototherapy bed most commonly called a “bilibed”. The greatest benefit of the fiber-optic panel is that the parent is able to hold the infant without interrupting treatment, thereby supporting parent-infant bonding. The benefit of the “bilibed” is that it covers more of the infants back due to its size. The invention is highly portable, has a dense coverage pattern, and intensity levels potentially exceeding 120 μW/cm²/nm (microwatts per centimeter squared per nanometer), which is nearly double the current output of a neonatal fiber optic panel or a phototherapy bed while supporting parent-infant bonding, as well as maintaining a high coverage area.

The use of light-emitting diodes (LEDs) is used in a preferred embodiment to deliver light directly to the neonate's skin. The LEDs are very small, very durable, and long lasting. As a result, the panel comprising the LEDs is portable, lightweight, comfortable, easy to use, and relatively inexpensive. LEDs deliver relatively high light intensity for their physical size and weight, with relatively low power consumption (e.g., 70 mW), and therefore have high efficiency (optical output power/electrical input power). They produce no harmful UV radiation and negligible heat (infrared). The LEDs may emit light of a wavelength suitable for treating hyperbilirubinemia in neonates, such as 420-500 nm, preferably between 440 and 480 nm. Each lens type LED emits light substantially all of its light throughout an included angle of about 90 to about 130 degrees and preferably throughout an angle of 90 to 110 degrees, however the viewable angle is not limited to these viewing angles. In one preferred embodiment, the invention will utilize LEDs that have a wavelength output between 450 and 470 nm, with a viewing angle of 130 degrees. It should be appreciated that minimal light is emitted at wider angles which is wasted energy adding to heat loss.

In a preferred embodiment, the present invention uses lens type LEDs. The LEDs that will be used are not limited to a specific type of LED. For example, low profile lens type LEDs or organic LEDs (OLED) can be utilized if the light output is sufficient to meet the output requirements for successful treatment. Additionally, the LEDs can be either surface mount or through hole mount. A phototherapy panel is made of a flexible material that incorporates the LEDs. The panel has a lining inside surface of reflective Mylar that reflects ambient light that is previously reflected from the neonate's skin. An external liner or plastic sheath seals the panel for hygienic purposes. For example, the liner may be crimped (heat-sealed), stitched, or molded to material. In the preferred embodiment, the external sheath or lining will not hinder the flexibility of the panel, nor will the material hinder the light output of the needed wavelength for successful treatment.

Because the panel directly contacts the jaundiced neonate, it is highly desirable for the panel to be capable of easy cleaning and sterilization. The material is preferred to be impervious to the solution, solvent, or gas used for cleaning or sterilization. All electronic circuitry, including the LEDs, will be fully encapsulated or potted within a flexible material, such as a silicone gel 14. The gel may be poured over the LEDs and molded to yield a flexible, top planar support surface on which the infant rests. The gel fully encapsulates the tops of the LEDs, the MYLAR reflective sheet, the flexible printed circuit, and the control circuits, thereby creating a single layer of flexible gel that encompasses the device completely. In the preferred embodiment, the power connection for either the AC or DC power supply will be the only exposed component of the circuitry. In the preferred embodiment, the power connection will be resistant to fluids, thus eliminating any threat of electrical shock for the patient when in use. The gel is preferred to be transparent or substantially transparent to the selected efficacious wavelength of the LEDs. The encapsulating or potting gel simplifies cleaning, and limits the surface area that can harbor bacteria, while protecting the LEDs from impact, liquids (from the infant, or otherwise), and cleaning materials and abrasives. Additionally, the panel liner may be coated with an anti-bacterial coating, such as MEDIGARD, manufactured by the Hydrogiene Corporation of San Diego, Calif. Medigard is formulated to kill a wide spectrum of bacteria, is resistant to cleaning and sterilization processes, and has a reputed four-year killing period. It can be coated in such a way as to be impossible to detach or destroy when applied to materials. Additionally, the panel cover may be disposable, thereby reducing the need for cleaning and sterilizing the liner.

As shown in FIG. 4, the tops of the plurality of the LEDs are fully encapsulated by the substantially transparent gel 14. A reflective sheet 18 surrounds the plurality of LEDs 10. The reflective sheet is below the tops 20 of the LEDs so that light from the LEDs is reflected toward the top planar surface 16 of the panel. The reflective sheet may have a plurality of holes formed therein that are spaced to accept the LEDs within the holes. The reflective sheet will have enough space around the perforations to allow both the light of the LEDs to pass through as well as to allow the flexible gel to pass through thus forming a continuous single layer of flexible encapsulating gel. The reflective sheet is preferred to be not be materially electrically conductive, and may be MYLAR having a silver or white colored top surface. In another embodiment, the flexible substrate's 22 top surface would be white in color thereby eliminating the need for a MYLAR reflective sheet. The flexible circuitry substrate 22 is flexible, and has conductors 24 printed thereon that provide power to the LEDs. The gel also surrounds the LEDs below the reflective sheet and above the flexible circuitry substrate, so that the LEDs are encapsulated, with no spaces or pockets having a material size around or above the LEDs. Additionally, the flexible substrate 22 can have holes strategically placed to allow more of the flexible encapsulating gel to pass through flexible substrate 22 to promote greater structural integrity. As shown in FIG. 5, which is an additional embodiment from the embodiment of FIG. 4, the plurality of the LEDs 10 are fully encapsulated by the substantially transparent gel 14. A reflective sheet 18 surrounds the plurality of LEDs. The reflective sheet is adjacent to and above the flexible circuitry substrate, so that light from the LEDs is reflected toward the top planar surface 16 of the panel. The reflective sheet is preferred to not be materially electrically conductive, and may be MYLAR having a silver or white colored top surface. The non-conductive reflective sheet may be in contact with the flexible circuitry substrate. The reflective sheet may have a plurality of holes formed therein that are spaced to accept the LEDs within the holes. The flexible circuitry substrate is flexible, and has conductors printed thereon that provide power to the LEDs. The gel is also present below the reflective sheet and the flexible circuitry substrate. All circuitry and the LEDs are encapsulated/potted by the gel, other than the power supply and leads 26 to the power supply. No spaces or pockets of material size are around or above the LEDs, other than perhaps air bubbles from the formation process. Lead 26 extends from the gel material and communicates with the LEDs by a printed circuit on the flexible circuitry substrate.

In the preferred embodiment, the device can use either surface mount or through-hole mount LEDs. Each panel is preferred to contain not more than 70 low profile lens type LEDs that do not exceed 2.5 mm in height. The LEDs are mounted on a flexible circuit substrate, and are connected to a circuit bus that communicates with the duty cycle and current regulator 8, and the power supply 10. This device is preferred to have an irradiance level of at least 80 μW/cm²/nm. The lens type LED's provide higher intensity levels than LEDs without lenses.

The power supply connection is preferred to be a waterproof or moisture-proof electrical plug that attaches the electrical cord to the power supply. The power cord may be removed for cleaning by unplugging the cord from the waterproof connector. Even though connection protrudes from the panel, cleaning solutions cannot enter the panel.

The power supply may be a standard portable power supply capable of generating a relatively low DC voltage, such as 9V, 5V or 3.3V provided by a battery, including a rechargeable battery. Low voltage is desirable for reducing the risk of electrocution for the neonate under treatment. Additionally, the preferred embodiment will also have an AC power adapter enabling longer treatment times without interruptions due to the replacement of batteries used in the DC power source. Appropriate circuitry is provided to control the current to the LEDs.

The current supplied to LEDs is not constant in one embodiment, but is rather pulsed, with a duty cycle of 5-95%. The potted circuitry contains appropriate electronic circuitry to control the timing and activation of LEDs. For example, a circuit 12 providing a direct current duty cycle and current regulator may be provided for intermittent operation. Operating LEDs at such a low duty cycle allows them to be overdriven, i.e., operated with a higher applied current than would be feasible with a constant current. The LEDs are not overdriven because the output of the lens type LED is far brighter than that of the surface mount type used by the current artwork device. This also alleviates the need for a heat sink to pull heat away from the device. The allowable ranges for duty cycle, forward current, and pulse width are determined by the operating characteristics of the particular LEDs that are used, according to information provided by the LED manufacturer. Another embodiment of the device would enable the power supply to have external controls that allow a person supervising the phototherapy to control the light intensity, frequency (i.e., pulse width), duty cycle, and color balance if multicolored LED arrays are used. It may instead have preprogrammed control circuitry that regulates the exposure time and other variables of the treatment.

The intermittent power may be supplied with any pulse width satisfying the LEDs¹ operating constraints. Intermittent operation at higher power provides significantly more efficient bilirubin photoconversion than does constant operation at lower power. Bilirubin is produced as a by-product of the break down of hemoglobin, which is at high levels after birth. After birth, excess hemoglobin is broken down in a short period of time thereby causing high bilirubin levels (hyperbilirubinemia). Bilirubin enters the skin in significant amounts when the bilirubin concentration is quite high, leading to the characteristic yellowish skin tone. Phototherapy is believed to affect bilirubin in the skin only. When the light is off, the bilirubin concentration in the skin gradually builds up, and it is then converted when the light is on again. Therefore, consistent phototherapy with high irradiance levels is necessary to reduce the overall bilirubin concentrations in the neonate.

The present invention allows the neonate to be fully covered during phototherapy via a gown or blanket, and therefore, the neonate is much more comfortable and less susceptible to hypothermia. The neonate may be wrapped in a blanket, with the present invention inside the blanket against the child's back. The device may also be placed inside clothing. The flexible casting allows deformation of the device as the child is held within a blanket, without damaging or adversely affecting the performance of the device.

In one embodiment, a power supply 8 includes a battery, which may be replaceable or rechargeable, that supplies power to LEDs. The battery or batteries may be held in a pocket or pack that is attached to the outside of the gel material or attached to the care givers belt or clothing. Appropriate circuitry for pulsing the LEDs will be encapsulated/potted in the gel material. The use of a battery makes the panel fully portable, allowing it to be used when the neonate is transported to and from, and within, the hospital, for example.

Treatment time using the phototherapy device of the present invention varies widely depending upon the conditions of the particular neonate, particularly the serum bilirubin level. However, treatment times are less than with prior methods, which provide much lower irradiance and less surface exposure. The LEDs may be arranged in various configurations. LEDs emitting wavelengths of light other than blue wavelengths may be used, and multiple different wavelengths may be used on the same panel. Phototherapy has been shown to cause a decrease in the endogenous levels of riboflavin, a natural photosensitizer. When irradiated by broad wavelength white or blue light, riboflavin produces singlet oxygen that may react with bilirubin and other organic compounds, causing photodamage and decreased riboflavin levels in developing neonates. Narrow wavelength blue to green LED light is less likely to be absorbed by endogenous photosensitizers and appears to produce fewer side effects, and thus LEDs with higher wavelengths (blue to green) may be included in the device of the present invention.

The device is preferred to be flexible in both the longitudinal and transverse direction. In this embodiment, a substantially transparent casting silicon plastic or gel is poured over the device to encase the circuitry, and is allowed to set thereby fully potting the LEDs, the printed circuitry, the reflective sheet, and the control circuitry. In particular, the LEDs are covered with the casing formed by the plastic or gel leaving no spaces, pockets, or channels in the panel. The casing serves as structural support to counter the weight of the patient, thereby preventing the LEDs from being damaged or moved out of proper alignment for maximum efficacy, as well as insuring patient safety and protection from the electrical circuitry. The encasing material is flexible, and allows the device to be deformed so as to follow contours of the infant's body, particularly as the infant is held.

A substantially transparent removable sheath, which is preferred to be non-latex, may contain the device. A cover 4 having a large mesh that will permit transmission of light through the mesh may be used in place of, or in addition to, the sheath. The sheath or cover may be removed and cleaned, or disposed of and replaced, for disinfection purposes.

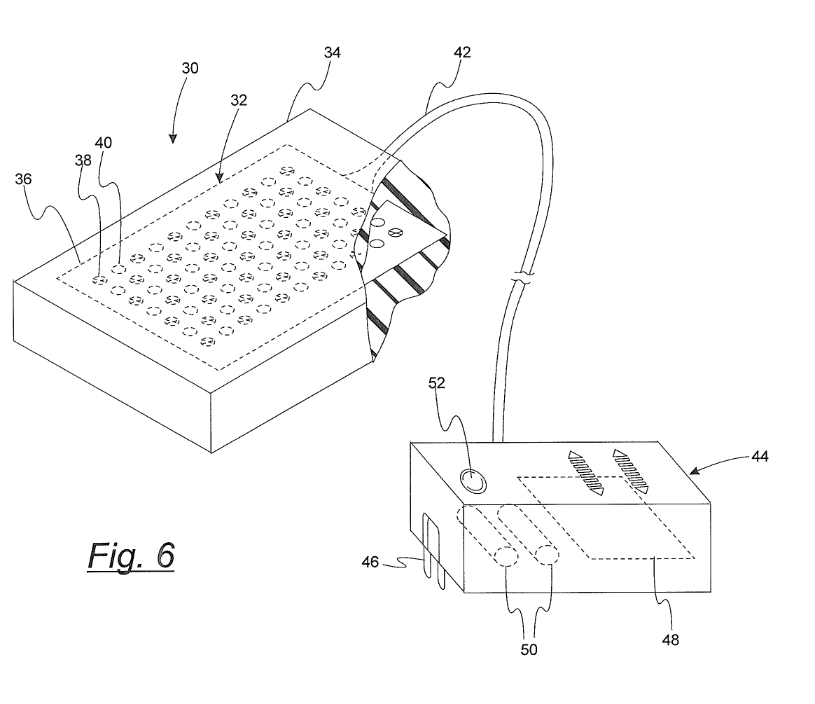

FIG. 6 illustrates an alternative embodiment of the invention. Phototherapy device 30 includes an LED array 32 potted within a polymeric mat 34. Preferably, the polymeric material is silicone, however, the invention is not limited to silicone resins and other transparent flexible resins could be used. The LED array 32 includes a flexible circuit board 36 having an upper side provided with a light reflective surface and a lower side providing a printed circuit board layout for powering a pair of arrays of LEDs 30 and 40. In the embodiment illustrated, half of the LEDs are part of array 38 are of one color and the other half of the LEDs are of a different color. More than two LED color arrays could be used if desired. Printed flexible circuit board 36 has a series of through holes to accommodate the leads of the LEDs, however, the invention is not limited to printed circuit boards having through holes and lens type LEDs. LED arrays can alternatively be made using surface mount LEDs.

Power supply cable 42 connects the printed circuit board 36 and the associated arrays of LEDs 30 and 40 to a power supply 44. Preferably, power supply 44 includes a retractable AC connector 46 which is connected to a control board 48 having a rectified circuit to generate DC power. Preferably, the power supply further includes a rechargeable DC battery 50 coupled to a charger circuit on the control board 48. The power supply further includes an on/off switch 52 and a power output level and a color output display. An input device enabling the power and color balance to be varied can alternatively provided on the exterior of the power supply 44 or preset using a non-user accessible input/output set by the medical supply device distributor pursuant to the physician's instruction. The power to the LED arrays 30 and 40 can be independently set using a pair of pulse width modulation circuits to provide the total power output and average color desired. Alternatively a single array of multicolor LEDs could be used to enable the color balance to be varied.

In use, when the baby is at home, the power supply 44 may be plugged into a AC wall outlet. When it is desired to move the baby away from the wall outlet, the power supply can be simply unplugged and the device operated in the DC mode drawing power from rechargeable batteries 50. Preferably, the power supply 44 will be small and compact, comparable to or smaller than the size of a deck of playing cards, so that it can be easily placed in a pocket of a caregiver holding the baby. Of course, a larger power supply could accommodate a large battery and longer use time in the DC mode of operation.

Preferably, control board 48 further includes a timer circuit so that the device can be rendered inoperative after some predetermined number of hours of use or after a predetermined period of time. For example, when a device is provided by a medical equipment supply company, the timer may be set for a period of time sufficient to treat a single infant, for example, 200 hours of use time, two weeks or both. After the time limit has been achieved, the device could be rendered inoperative. This will ensure that the device is in fact only used to treat the single patient for which the device is prescribed. Alternatively, or in combination with a single use time limit, a total elapsed time limit may be established so the device can be taken out of service once it has achieved its useful life, for example 10,000 hours or the like. In this way, the risk of an end of useful life failure of the LED or the power supply of a phototherapy device while in a rental program can be greatly reduced. Preferably, the total elapsed time information can be read and the individual patient time limit can be reset by the medical equipment distributor prior to being provided to a new patient. The LED total life timer continues to accumulate hours in each successive use and is preferably non-resettable.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A phototherapy device for treating neonatal hyperbilirubinemia, comprising:
flexible circuitry, said flexible circuitry comprising a plurality of light emitting diodes mounted in a pattern,
a panel formed of a flexible material surrounding and encasing the flexible circuitry and the plurality of light emitting diodes, and
a power supply for powering said LEDs.

2. A phototherapy device for treating neonatal hyperbilirubinemia as described in claim 1, wherein said flexible circuitry is completely surrounded by and is potted in said flexible material.

3. A phototherapy device for treating neonatal hyperbilirubinemia as described in claim 1, wherein said each of said light emitting diodes that comprises said plurality of light emitting diodes are surrounded by a sheet of reflective material that is encased in said flexible material, and wherein said reflective material is below a top of each of said light emitting diodes that comprises said plurality of light emitting diodes.

4. A phototherapy device for treating neonatal hyperbilirubinemia as described in claim 1, wherein said each of said light emitting diodes that comprises said plurality of light emitting diodes are mounted on a flexible substrate that has a white reflective surface, and is fully potted.

5. A phototherapy device for treating neonatal hyperbilirubinemia as described in claim 1, wherein said each of said light emitting diodes that comprises said plurality of light emitting diodes are surrounded by a sheet of reflective material that is encased in said flexible material, and wherein said reflective material is below a top of each of said light emitting diodes that comprises said plurality of light emitting diodes, and wherein said reflective material is positioned over said flexible circuitry.

6. A phototherapy device for treating neonatal hyperbilirubinemia as described in claim 1, wherein said plurality of light emitting diodes are lens type light emitting diodes.

7. A phototherapy device for treating neonatal hyperbilirubinemia as described in claim 1, wherein said plurality of light emitting diodes are surface mount light emitting diodes.

8. A phototherapy device for treating neonatal hyperbilirubinemia as described in claim 1, wherein said plurality of light emitting diodes are through hole mount light emitting diodes.

9. A phototherapy device for treating neonatal hyperbilirubinemia as described in claim 1, wherein said flexible material comprises silicone gel.

10. A phototherapy device for treating neonatal hyperbilirubinemia as described in claim 1, wherein said flexible material is substantially transparent to an efficacious wave length of light emitted by said plurality of light emitting diodes.

11. A phototherapy device for treating neonatal hyperbilirubinemia as described in claim 1, wherein said flexible material is covered by a sheath, and wherein said sheath is substantially transparent to an efficacious wave length of light emitted by said plurality of light emitting diodes.

12. A phototherapy device for treating neonatal hyperbilirubinemia as described in claim 1, wherein said plurality of light emitting diodes is controlled by a circuit that operates said plurality of light emitting diodes on an intermittent basis.

13. A phototherapy device for treating neonatal hyperbilirubinemia as described in claim 9, wherein said flexible circuitry and said circuit that operates said plurality of light emitting diodes on an intermittent basis is surrounded by and is encased in said flexible material.

14. A phototherapy device for treating neonatal hyperbilirubinemia as described in claim 9, wherein said flexible circuitry, the LEDs, the control circuitry, and the reflective sheet are all fully potted together.

15. A phototherapy device for treating neonatal hyperbilirubinemia as described in claim 9, wherein the device is potted in a single layer of transparent silicone gel.
